(19) United States
(12) Patent Application Publication
(10) Pub. No.: US 2024/0177981 A1
(43) Pub. Date: May 30, 2024

(54) METHOD AND DEVICE FOR PROCESSING MASS SPECTROMETRY DATA

(71) Applicant: SHIMADZU CORPORATION, Kyoto-shi (JP)

(72) Inventors: Yoshihiro KUNIMURA, Kyoto-shi (JP); Kosuke UCHIYAMA, Kyoto-shi (JP)

(73) Assignee: SHIMADZU CORPORATION, Kyoto-shi (JP)

(21) Appl. No.: 18/473,700

(22) Filed: Sep. 25, 2023

(30) Foreign Application Priority Data
Nov. 29, 2022 (JP) 2022-190770

(51) Int. Cl. H01J 49/00

(52) U.S. Cl. CPC H01J 49/0036

(57) ABSTRACT

Provided is a method for processing mass spectrometry data by which the task of analyzing data of mass spectra acquired by a mass spectrometric analysis of an analysis target sample can be efficiently performed. The method includes the steps of: preparing data of a plurality of MS/MS spectra for each of one or more compounds contained in a sample, each of the MS/MS spectra acquired by an MS/MS scan measurement using each of a plurality of different precursor ions originating from the same compound (Steps 1-3); and creating data of a merged MS/MS spectrum for each of the one or more compounds by merging the data of the plurality of MS/MS spectra acquired by the MS/MS scan measurement using the plurality of precursor ions originating from the compound concerned (Step 6).

### TECHNICAL FIELD

The present invention relates to a method and device for processing data obtained by a mass spectrometric analysis of a compound contained in a sample.

### BACKGROUND ART

With the aim of treating genetic disorder or similar diseases, efforts have been made to develop nucleic acid drugs which use nucleic acids as drugs. Nucleic acid drugs are chemically synthesized molecules whose molecular masses range from thousands to tens of thousands. Those molecules have a chain structure in which tens of nucleotides, such as adenine (A), guanine (G), cytosine (C), uracil (U) and thymine (T) which constitute DNAs and RNAs, are connected via linkers (for example, see Non Patent Literature 1).

Chemically synthesized nucleic acids or peptides may possibly contain various foreign compounds originating from materials used for the synthesis, or impurities produced in the synthesizing process, or other substances, along with the intended nucleic acids or peptides (“target compounds”). Therefore, after a nucleic acid or peptide has been chemically synthesized, the sample is examined by chromatograph mass spectrometry to determine the presence or absence of not only the target compound but also foreign compounds, and to identify the foreign compound if a foreign compound has been found.

There are a wide variety of compounds that can be contained in a sample of a nucleic acid or peptide. Therefore, for a chromatograph mass spectrometric analysis of this type of sample, the data dependent acquisition (DDA), in which the compounds separated from each other by the column of a chromatograph are exhaustively measured, is often used to acquire mass spectrometry data. In the DDA, a process is repeatedly performed in which an MS scan measurement of ions generated from compounds in a sample is initially performed, and if an ion having an intensity exceeding a previously determined threshold has been detected, an MS/MS scan measurement with the detected ion as the precursor ion is subsequently performed. The MS scan measurement is a measurement in which ions generated from the compounds in the sample are detected with scanning mass-to-charge ratio within a predetermined mass-to-charge-ratio range. The MS/MS scan measurement is a measurement in which product ions generated by the dissociation of a precursor ion are detected with scanning mass-to-charge ratio within a predetermined mass-to-charge ratio.

In a normal mode of DDA, the MS/MS scan measurement is repeatedly performed during a period of time in which target compounds or foreign compounds are introduced from the column of the chromatograph into the mass spectrometer, whereby a set of data corresponding to one MS/MS spectrum is obtained with each execution of the measurement. After the completion of the measurement, the sets of data corresponding to a plurality of MS/MS spectra obtained for precursor ions having the same mass-to-charge ratio are merged into data corresponding to one MS/MS spectrum by an appropriate method, e.g., by calculating an average of the intensities of the mass peaks at the same mass-to-charge ratio.

For example, in the case of a nucleic acid, which has a chain structure consisting of tens of nucleotides, such as adenine (A), guanine (G), cytosine (C), uracil (U) and thymine (T), it has been commonly known that the dissociation of a precursor ion occurs at a bonding site (“linker”) of those nucleotides. Therefore, it is possible to theoretically calculate the mass-to-charge ratios of the fragment ions which will be generated from a precursor ion in an MS/MS scan measurement. The mass-to-charge ratios of fragment ions originating from a foreign compound can also be calculated from related information, such as the kind of reagent used for the chemical synthesis. By comparing those theoretically calculated values of the mass-to-charge ratio with the mass-to-charge ratios of the mass peaks included in the data of the merged MS/MS spectrum, a fragment ion corresponding to each mass peak is determined, and the compound is identified.

### CITATION LIST

### Non Patent Literature

Non Patent Literature 1: “Kakusan-Iyakuhin No Gousei Kakunin —MALDI-TOF MS Ni Yoru Jinsoku Kan-i Hairetsu Kakunin-(Synthesis Check of Nucleic Acid Drugs —Easy and Quick Check of Sequence by MALDI-TOF MS-)”, [online], August 2017, Shimadzu Corporation, [accessed on Nov. 14, 2022], the Internet

### SUMMARY OF INVENTION

### Technical Problem

Ionizing a nucleic acid or peptide produces ions having various numbers of charges. When ions (precursor ions) which are identical in molecular structure yet different in number of charges are dissociated, each precursor ion may be dissociated at a different position and produce different fragments, so that data of an MS/MS spectrum with a different pattern are obtained. Therefore, by analyzing the data of the MS/MS spectrum obtained for each of the precursor ions having various number of charges, the accuracy of the identification of the compound can be improved.

On the other hand, a sample of a nucleic acid or peptide contains hundreds or even thousands of compounds (target and foreign compounds). If data of a plurality of MS/MS spectra are prepared for each of the large number of compounds, the number of sets of data will be enormous. Analyzing those data will require a considerable amount of time and labor.

Although the description thus far has been concerned with the example of acquiring mass spectrometry data by DDA using a chromatograph mass spectrometer for a sample containing a nucleic acid or peptide, the previously described problem similarly occurs in the case of acquiring mass spectrometry data by a measurement method different from DDA, as in the case of acquiring mass spectrometry data using an independent mass spectrometer, or in the case of performing an MS/MS scan measurement for a precursor ion having a previously determined mass-to-charge ratio for each compound.

The problem to be solved by the present invention is to provide a method and device for processing mass spectrometry data by which the task of analyzing data of mass spectra acquired by a mass spectrometric analysis of an analysis target sample can be efficiently performed.

### Solution to Problem

The method for processing mass spectrometry data according to the present invention developed for solving the previously described problem includes the steps of:
preparing data of a plurality of MS/MS spectra for each of one or more compounds contained in a sample, each of the plurality of MS/MS spectra being acquired by an MS/MS scan measurement using each of a plurality of different precursor ions originating from the same compound; and
creating data of a merged MS/MS spectrum for each of the one or more compounds by merging the data of the plurality of MS/MS spectra acquired by the MS/MS scan measurements using the plurality of precursor ions originating from the compound concerned.

The device for processing mass spectrometry data according to the present invention developed for solving the previously described problem includes:
a storage section holding data of a plurality of MS/MS spectra for each of one or more compounds contained in a sample, each of the plurality of MS/MS spectra being acquired by an MS/MS scan measurement using each of a plurality of different precursor ions originating from the same compound; and
a merged MS/MS spectrum data creator configured to create data of a merged MS/MS spectrum for each of the one or more compounds by merging the data of the plurality of MS/MS spectra acquired by the MS/MS scan measurements using the plurality of precursor ions originating from the compound concerned.

### Advantageous Effects of Invention

In the method and device for processing mass spectrometry data according to the present invention, data of a plurality of MS/MS spectra acquired by an MS/MS scan measurement using a plurality of different precursor ions originating from the same compound are prepared for each of the one or more compounds contained in a sample. This task may be carried out by acquiring the data by actual measurements, or by retrieving, from a storage section, the data acquired beforehand and stored in that section. The plurality of different precursor ions can include, for example, ions identical in mass number and different in number of charges, adduct ions, isotopic ions, dehydrated ions and fragment ions. By performing an MS/MS scan measurement using each ion as a precursor ion, data of a plurality of MS/MS spectra having different patterns can be obtained.

In the present invention, data of a plurality of MS/MS spectra acquired in the previously described manner by an MS/MS scan measurement using precursor ions originating from the same compound are merged to obtain data of one merged MS/MS spectrum. The merging of the MS/MS spectra can be performed, for example, by calculating an average or total of the intensities of mass peaks having the same mass-to-charge ratios. The method and device for processing mass spectrometry data according to the present invention only require checking the data of the merged MS/MS spectra whose number is equal to that of the compounds contained in the sample. Accordingly, the analyzing task can be more efficiently performed than by the conventional technique.

### BRIEF DESCRIPTION OF DRAWINGS

FIG. 1 is a diagram showing the configuration of the main components of a liquid chromatograph mass spectrometer including one embodiment of the device for processing mass spectrometry data according to the present invention.

FIG. 2 is a flowchart according to one embodiment of the method for processing mass spectrometry data according to the present invention.

FIG. 3 is a model diagram showing the flow of DDA in the present embodiment.

FIG. 4 is a diagram illustrating the classification of precursor ions in the present embodiment.

FIG. 5 is a diagram illustrating an MS scan measurement and an MS/MS scan measurement performed in the present embodiment.

FIG. 6 is a diagram illustrating data of MS/MS spectra acquired in the present embodiment.

FIG. 7 is an example of a merged MS/MS spectrum created by calculating an average of the intensities of mass peaks.

FIG. 8 is an example of a merged MS/MS spectrum created by extracting the highest intensity of the mass peak.

FIG. 9 is an example of an analysis result display screen in the present embodiment.

### DESCRIPTION OF EMBODIMENTS

An embodiment of the method and device for processing mass spectrometry data according to the present invention is hereinafter described with reference to the drawings.

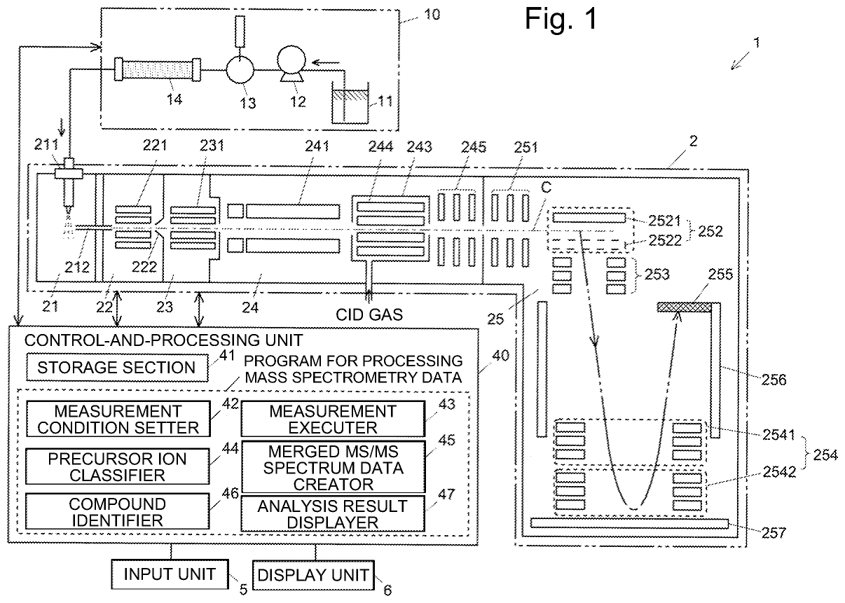

FIG. 1 is a diagram showing the configuration of the main components of a liquid chromatograph mass spectrometer 1 including the device for processing mass spectrometry data according to the present embodiment. The liquid chromatograph mass spectrometer 1 according to the present embodiment includes a liquid chromatograph 10, mass spectrometer 20, and control-and-processing unit 40 configured to control the operations of those devices. The control-and-processing unit 40 corresponds to an embodiment of the device for processing mass spectrometry data according to the present invention.

The liquid chromatograph 10 includes: a mobile phase container 11 in which a mobile phase is stored; a pump 12 for drawing the mobile phase and supplying it at a constant flow rate; an injector 13 for injecting a liquid sample into the mobile phase; and a column 14 for separating compounds contained in a liquid sample. In the case of continuously analyzing a plurality of liquid samples, an autosampler (not shown) is additionally provided, and a plurality of liquid samples set in this autosampler are sequentially injected from the injector 13.

The mass spectrometer 20 includes an ionization chamber 21 and a vacuum chamber. The vacuum chamber is evacuated with vacuum pumps (not shown). The vacuum chamber internally has a first intermediate vacuum chamber 22, second intermediate vacuum chamber 23, third intermediate vacuum chamber 24 and analysis chamber 25 sequentially arranged from the ionization chamber 21 and configured as a multistage differential pumping system in which the degrees of vacuum of those chambers are increased in the mentioned order.

In the ionization chamber 21, an electrospray ionization (ESI) probe 211 configured to spray a sample solution while imparting electric charges to the same solution is installed. The ionization chamber 21 communicates with the first intermediate vacuum chamber 22 in the next stage through a capillary tube 212.

The first intermediate vacuum chamber 22 contains an ion guide 221 formed by a plurality of rod electrodes. The ion guide 221 is configured to converge the flight path of ions along the ion beam axis C. The first intermediate vacuum chamber 22 is separated from the second intermediate vacuum chamber 23 by a skimmer 222 having a small hole at its apex.

The second intermediate vacuum chamber 23 contains an ion guide 231 formed by a plurality of rod electrodes. Similar to the ion guide 221, the ion guide 231 is configured to converge the flight path of ions along the ion beam axis C. The second intermediate vacuum chamber 23 is separated from the third intermediate vacuum chamber 24 by a partition wall in which a small hole is formed.

The third intermediate vacuum chamber 24 contains: a quadrupole mass filter 241 having pre-rod electrodes and main-rod electrodes for separating ions according to their mass-to-charge ratios; a collision cell 243 containing a multipole ion guide 244; and an ion guide 245 formed by a plurality of ring electrodes. The collision cell 243 can be internally supplied with a collision induced dissociation (CID) gas, such as argon or nitrogen, from a gas source (not shown) as needed.

The analysis chamber 25 contains an ion guide 251 formed by a plurality of ring electrodes, an orthogonal acceleration electrode 252 formed by a push-out electrode 2521 and a pulling electrode 2522, as well as a second acceleration electrode 253, reflectron 254, flight tube 256, back plate 257 and ion detector 255. The push-out electrode 2521 is a plate-shaped electrode, while the pulling electrode 2522 is an electrode shaped like a plate in its entirety, with an ion-passing area formed at its center. The second acceleration electrode 253 has a plurality of ring-shaped electrodes and a slit plate located behind them. The reflectron 254 consists of a first reflectron 2541 and a second reflectron 2542 both of which are a plurality of ring-shaped electrodes. The flight tube 256 is a cylindrical electrode. The back plate 257 is a plate-shaped electrode.

In the mass spectrometer 20, an MS scan measurement and an MS/MS scan measurement can be performed. In the MS scan measurement, the quadrupole mass filter 241 performs no selection of ions (i.e., it is not enabled to function as a mass filter); it allows all ions generated from a sample to enter the analysis chamber 25 in a non-selective manner.

The ions which have entered the analysis chamber 25 are converged by the ion guide 251 along the ion beam axis C and enter the space between the push-out electrode 2521 and the pulling electrode 2522 (“orthogonal acceleration space”).

The orthogonal acceleration electrode 252 is supplied with a pulsed voltage with a constant frequency. By the application of this pulsed voltage, an electric field for deflecting the flight direction of ions in the orthogonal direction to the ion beam axis C (in the direction from the push-out electrode 2521 to the pulling electrode 2522) is created within the orthogonal acceleration space. The ions whose fight direction has been deflected by the orthogonal acceleration electrode 252 are given a specific amount of kinetic energy from the acceleration electric field formed by the application of voltages to the second acceleration electrode 253. The accelerated ions fly in a returning path defined by the reflectron 254, flight tube 256 and back plate 257, before striking the ion detector 255. Since an ion having a smaller mass-to-charge ratio flies faster, the ions are separated from each other according to their respective mass-to-charge ratios while flying in the flight path, to ultimately hit and be detected by the ion detector 255 in ascending order of mass-to-charge ratio.

In the MS/MS scan measurement, the quadrupole mass filter 241 also performs a selection of ions (it is enabled to function as a mass filter). At the quadrupole mass filter 241, only an ion designated as a precursor ion is allowed to pass through. Meanwhile, a CID gas is supplied into the collision cell 243, and the precursor ion is accelerated and introduced into this cell so as to make the precursor ion collide with the CID gas and thereby promote the fragmentation of the precursor ion. The product ions resulting from the fragmentation of the precursor ion are converged by the ion guide 245 so that they fly along the ion beam axis C and enter the analysis chamber 25. As in the case of the MS scan measurement, the product ions which have entered the analysis chamber 25 are made to fly in the returning path within the flight space, and the ions which have been separated from each other according to their mass-to-charge ratios during their flight are sequentially detected with the ion detector 255.

The control-and-processing unit 40 has a storage section 41. The storage section 41 holds a compound database in which measurement conditions for various compounds are described (e.g., the retention time and MS/MS scan measurement conditions), as well as information concerning various conditions for performing DDA (e.g., the intensity threshold and mass scan range).

The control-and-processing unit 40 includes, as its functional blocks, a measurement condition setter 42, measurement executer 43, precursor ion classifier 44, merged MS/MS spectrum data creator 45, compound identifier 46, and analysis result displayer 47. The control-and-processing unit 40 is actually a personal computer, which is enabled to act as the aforementioned functional components by running a dedicated program previously installed on the same computer. Additionally, an input unit 5 including a mouse, keyboard and other devices, as well as a display unit 6 including a liquid crystal display and other devices, are connected to the control-and-processing unit 40.

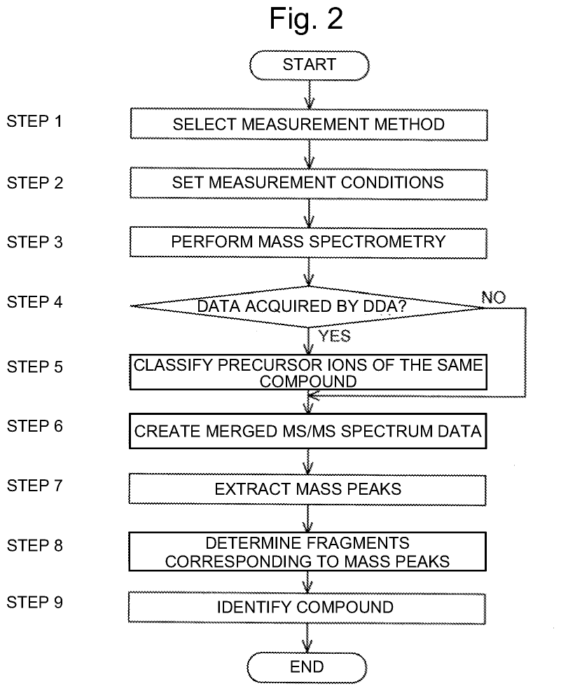

The flow of the analysis using the device for processing mass spectrometry data according to the present embodiment is hereinafter described. FIG. 2 is a flowchart of one embodiment of the method for processing mass spectrometry data according to the present invention. Although the following description is concerned with an example in which data of MS/MS spectra are acquired by an actual measurement of a sample, it is alternatively possible to prepare the data of MS/MS spectra by retrieving data of MS/MS spectra acquired by a previous measurement and stored in the storage section.

A user issues a command to initiate an analysis of a sample. Then, the measurement condition setter 42 displays target MS/MS and DDA as measurement methods on the screen of the display unit 6, prompting the user to select one method (Step 1).

After the measurement method has been selected by the user, the measurement condition setter 42 prompts the user to set measurement conditions necessary for performing the selected measurement method (Step 2). Specifically, when the target MS/MS has been selected by the user, the measurement condition setter 42 reads a compound list from the compound database stored in the storage section 41 and allows the user to select compounds which should be the targets of the measurement. After the compounds have been selected, the measurement condition setter 42 reads the measurement conditions for those compounds (such as the retention time, mass-to-charge ratio of the precursor ion, and mass scan range for the MS/MS scan measurement) and sets the measurement conditions. When the DDA has been selected by the user, the measurement condition setter 42 reads the related measurement conditions from the storage section 41 and sets those measurement conditions, such as the mass scan range for the MS scan measurement, measurement intensity value (threshold) to be used as the reference for selecting a precursor ion for which the MS/MS scan measurement should be performed, and mass scan range for the MS/MS scan measurement. It is also possible to additionally allow the user to modify these measurement conditions as needed.

After the measurement method and the measurement conditions have been determined, the user sets the sample and issues a command to initiate the measurement. Then, the measurement executer 43 performs a mass spectrometric analysis of the sample based on the measurement conditions set by the measurement condition setter 42 (Step 3). In the case of the target MS/MS, the MS/MS scan measurement of a compound selected as the target is repeatedly performed within the retention time of that compound. If there are two or more compounds whose retention times overlap each other, the MS/MS scan measurements for those compounds are sequentially and repeatedly performed during the overlapping period of time.

In the case of the DDA, an MS scan measurement over a mass-to-charge-ratio range specified in the measurement conditions is initially performed to acquire MS spectrum data. Subsequently, it is determined whether or not the MS spectrum data includes a mass peak whose intensity is equal to or higher than a threshold specified in the measurement conditions. If no mass peak having an intensity equal to or higher than the threshold has been found, the MS scan measurement is performed once again. If a mass peak having an intensity equal to or higher than the threshold has been found, an ion having the mass-to-charge ratio of that mass peak is selected as the precursor ion and an MS/MS scan measurement is performed within the mass-to-charge-ratio range specified in the measurement conditions, to acquire data of MS/MS spectra. If two or more mass peaks whose intensities are equal to or higher than the threshold have been found in the data of one MS spectrum, each of the ions having the mass-to-charge ratios of those mass peaks is selected as the precursor ion for performing the MS/MS scan measurement, to acquire a set of MS/MS spectrum data for each precursor ion. It should be noted that, when an MS/MS scan measurement is performed, ions whose mass-to-charge ratios fall within a range having a slight width (“mass window”; e.g., +1 to a few Da) centered on the mass-to-charge ratio of the precursor ion are selected as precursor ions in the quadrupole mass filter 241. This allows isotopic ions to be simultaneously selected as the precursor ions for the MS/MS scan measurement.

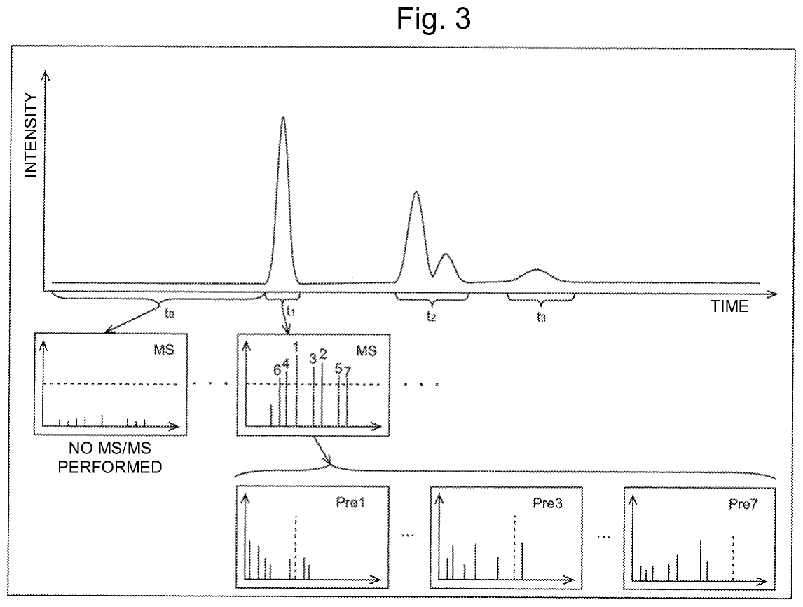

FIG. 3 is a model diagram showing the flow of the DDA. The upper section of FIG. 3 is the total ion current chromatogram (TICC) showing the temporal change in total ion intensity in the MS scan measurement. The TICC represents the temporal change in the amount of compound in the sample introduced into the mass spectrometer 20. Within time period to, no compound in the sample is practically introduced into the mass spectrometer 20, whereas some compounds in the sample are introduced into the mass spectrometer 20 within time periods t₁, t₂ and t₃. FIG. 3 also schematically shows examples of MS spectra (middle section) and MS/MS spectra (lower section). The kinds and quantities of compounds to be subjected to the mass spectrometry change with time within each time period t₁, t₂ or t₃, and the positions and intensities of the mass peaks which appear in the MS/MS spectra change accordingly.

After the beginning of the measurement, no mass peak which exceeds the threshold initially appears, and therefore, the MS scan measurement is repeatedly performed (for time period to) until a compound in the sample is introduced into the mass spectrometer 20. When a compound in the sample begins to be introduced into the mass spectrometer 20 (time period t₁), a mass peak whose intensity exceeds the threshold begins to appear in the MS spectrum data. In the example shown in FIG. 3 (the right MS spectrum shown in the middle section), there are seven mass peaks (1-7) whose intensities exceed the threshold. Therefore, each of the ions whose mass-to-charge ratios correspond to these seven mass peaks (1-7) is selected as the precursor ion, and an MS/MS scan measurement using that precursor ion is performed one time. The MS/MS scan measurements of the seven precursor ions are sequentially performed, for example, in descending order of intensity, or in descending (or ascending) order of mass-to-charge ratio. In the example of FIG. 3, the MS/MS scan measurements are sequentially performed in descending order of intensity, i.e., from precursor ion 1 to 7, and a set of data of an MS/MS spectrum is acquired for each precursor ion (lower section in FIG. 3). “Pre1” through “Pre7” mean that those MS/MS spectra are related to precursor ions 1 through 7, respectively. The peak indicated by the broken line in each MS/MS spectrum indicates the position (mass-to-charge ratio) of the related precursor ion. After the MS/MS scan measurements for all of the seven precursor ions have been completed, the MS scan measurement is performed once again, and the previously described process steps are similarly repeated.

The data of the mass spectra (MS spectra and MS/MS spectra) acquired by the previously described measurements (target MS/MS or DDA) are sequentially stored in the storage section 41 along with the time of the acquisition of the data. As for the data of the MS/MS spectra, the information of the mass-to-charge ratio of the precursor ion is additionally related to the data and stored in the storage section 41.

After the completion of the measurement, if the data of the MS/MS spectra are data acquired by DDA (“YES” in Step 4), the precursor ion classifier 44 examines the data of the MS spectrum which was acquired in each time period (t₁, t₂ or t₃) and used as the basis for the execution of the MS/MS scan measurement (i.e., an MS spectrum in which at least one mass peak with the intensity exceeding the threshold was present), and reads the data if the MS spectrum has a plurality of mass peaks whose intensities exceed the threshold.

Based on the mass-to-charge ratios of the mass peaks included in the read data of the MS spectrum, the precursor ion classifier 44 determines whether or not the ions corresponding to those mass peaks have originated from the same compound. Specifically, it identifies a set of mass peaks whose precursor ions can be expressed as [M+nH]ⁿ⁺ (where n is an integer equal to or greater than one) using a common value of M and different values of n, and classifies those mass peaks as originating from the same compound. In other words, a group of ions which are identical in mass number and different in number of charges are classified as originating from the same compound.

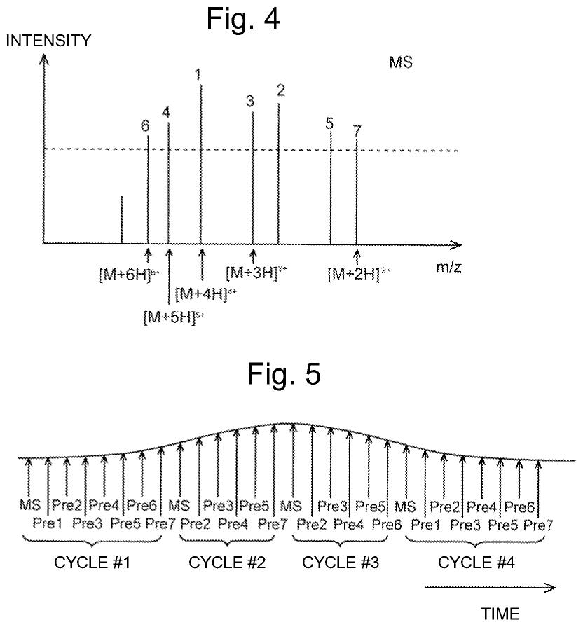

FIG. 4 shows one example. The MS spectrum shown in FIG. 4 is the same as the MS spectrum shown in the middle section of FIG. 3. This MS spectrum has seven mass peaks whose intensities exceed the threshold, of which five mass peaks are bivalent through hexavalent ions which satisfy the aforementioned condition. Accordingly, the precursor ion classifier 44 classifies those five ions as originating from the same compound (Step 5). In the example of FIG. 4, there is only one compound for which a plurality of ions are classified. Within a time period during which a plurality of compounds are simultaneously subjected to the mass spectrometry, it is possible that the mass peaks located in one MS spectrum include two or more groups of mass peaks respectively classified as two or more compounds.

If the data of the MS/MS spectra are data acquired by target MS/MS (“NO” in Step 4), the compound from which the MS/MS spectra were acquired is already known since those MS/MS spectra were acquired under specific measurement conditions previously determined for that compound. Accordingly, the previously described processing by the precursor ion classifier 44 should be omitted.

After the previously described processing for the MS spectrum acquired in each time period has been completed, the merged MS/MS spectrum data creator 45 merges the data of a plurality of MS/MS spectra originating from the same compound into one MS/MS spectrum (Step 6). In this processing, the mass peaks in the MS/MS spectra are compared to locate mass peaks having the same mass-to-charge ratio, extract a mass peak having the highest intensity among those mass peaks, and combine the extracted mass peaks into data of one MS/MS spectrum.

For example, one case is hereinafter described in which the measurement method was DDA, in which the MS scan measurement and MS/MS scan measurement were performed at the timing shown in FIG. 5 within time period t₁. In FIG. 5, the annotation “MS” stands for an MS scan measurement, while “Pre1” through “Pre7” mean MS/MS scan measurements for precursor ions 1-7, respectively. For simplicity, the following description concerning FIG. 5 assumes that one target compound and two foreign compounds are being measured within time period t₁ in which a single peak is appeared. In practice, it is possible that a plurality of target compounds and a considerable number of foreign compounds are simultaneously subjected to the mass spectrometry (as in time period t₂).

As shown in FIG. 5, when a mass peak exceeding the threshold has been detected in the MS scan measurement, an MS/MS scan measurement in which the ion corresponding to that mass peak is selected as the precursor ion is performed. Even when there is no change in the kinds of compounds subjected to the mass spectrometry (target and foreign compounds), a change may possibly occur in the intensities of the ions detected over the threshold. In the example shown in FIG. 5, the intensities of ions 1-7 exceed the threshold in the first and fourth MS scan measurements (cycles #1 and #4), so that the MS/MS scan measurement is performed for each of those ions. On the other hand, only the intensities of ions 2-4, 5 and 7 exceed the threshold in the second MS scan measurement (cycle #2), while only the intensities of ions 2-6 exceed the threshold in the third MS scan measurement (cycle #3), so that only the MS/MS scan measurement using those ions as precursor ions are performed.

The amounts of compounds subjected to the measurement also change with time. Therefore, even when an MS/MS scan measurement is performed for the same precursor ion, the intensities of the mass peaks in the MS/MS spectrum change depending on the timing of the measurement.

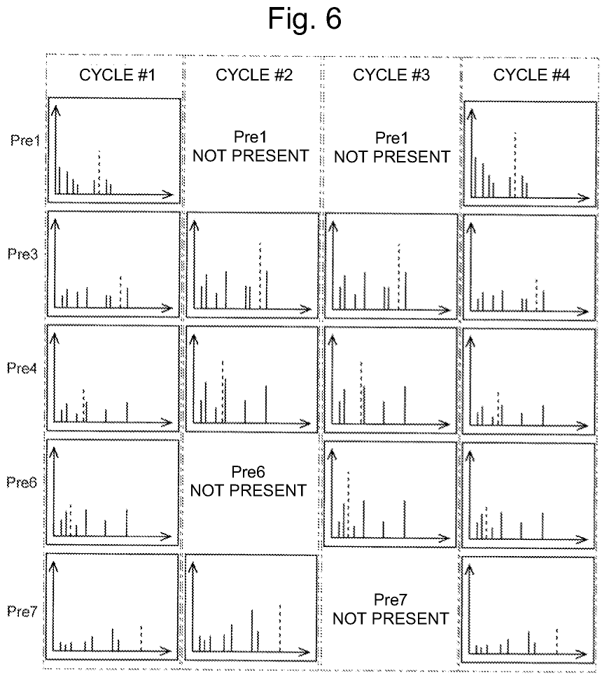

FIG. 6 shows an example of the MS/MS spectra acquired by the MS/MS scan measurement shown in FIG. 5. It should be noted that FIG. 6 shows only the MS/MS spectra of the precursor ions 1, 3, 4, 6 and 7 which have been classified as originating from the same compound and for which the data of the merged MS/MS spectra are to be created. In this example, four cycles of measurements are performed, with each cycle including an MS scan measurement and MS/MS scan measurements. Cycles #2 and #3 of the measurements are performed around the peak in the total ion current chromatogram (TICC), i.e., within a time period in which a large amount of compound was subjected to the mass spectrometry. By comparison, the amount of compound subjected to the mass spectrometry in cycles #1 and #4 is small since these cycles are close to the beginning and ending portions of the peak, respectively. Therefore, the intensities of the mass peaks in the MS/MS spectra acquired by the MS/MS scan measurements performed in cycles #1 and #4 are lower than those of the mass peaks in the MS/MS spectra acquired by the MS/MS scan measurements performed in cycles #2 and #3.

One method for creating a merged MS/MS spectrum for one compound is to calculate an average of the intensities of the mass peaks in a plurality of MS/MS spectra. When this method is employed in the example shown in FIGS. 5 and 6, the total intensity of the mass peaks at each mass-to-charge ratio should be divided by 16 (the total number of MS/MS spectra). Since the MS/MS scan measurement for precursor ion 1 were only performed in cycles #1 and #4, the average intensities of the mass peaks which only appear in the MS/MS spectra of precursor ion 1 will be lower than those of the mass peaks which appear in the MS/MS spectra acquired in all cycles, as with precursor ions 3 and 4. The same also applies in the case of precursor ions 6 and 7 for which the MS/MS scan measurements were performed three times each.

Additionally, the timings of the execution of the MS/MS scan measurement for precursor ion 1 are all within the beginning and ending portions of the TICC peak. The amount of compound subjected to the mass spectrometry within these portions is originally small, and the intensities of the mass peaks in the MS/MS spectrum acquired in each MS/MS scan measurement are also low. Therefore, when an average of the intensities of the mass peaks is calculated, the intensities of the mass peaks which only appear in the MS/MS spectra acquired in the MS/MS scan measurement for precursor ion 1 will be extremely low. On the other hand, the intensities of the mass peaks which appear regardless of the kind of precursor ion will be relatively higher.

Although the mode in which the data of a merged MS/MS spectrum are created by calculating an average of the intensities of mass peaks certainly falls within the scope of the present invention, a more preferable mode is adopted in the present embodiment, in which the data of a merged MS/MS spectrum are created by extracting the highest value of the intensity of the mass peak at each mass-to-charge ratio. In order to allow for a slight amount of mass error that can occur in mass spectrometry, a certain amount of allowance is given in the creation of the data of the merged MS/MS spectrum; for example, mass peaks whose difference in mass-to-charge ratio is equal to or smaller than 1 Da should be considered to be mass peaks whose mass-to-charge ratios are practically the same.

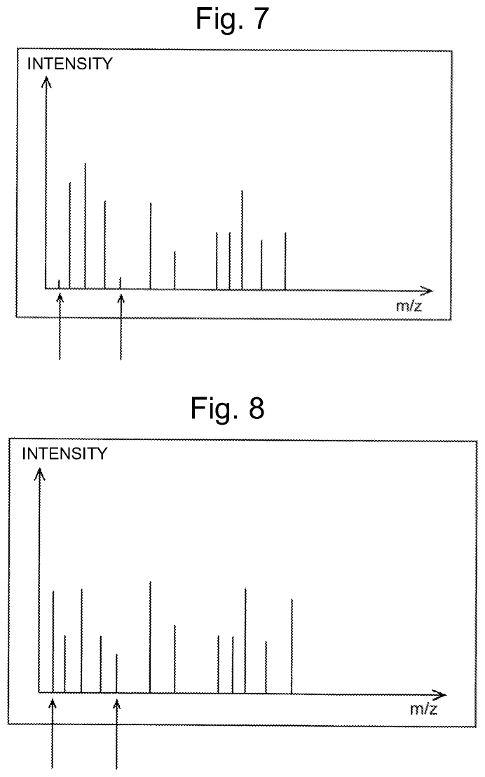

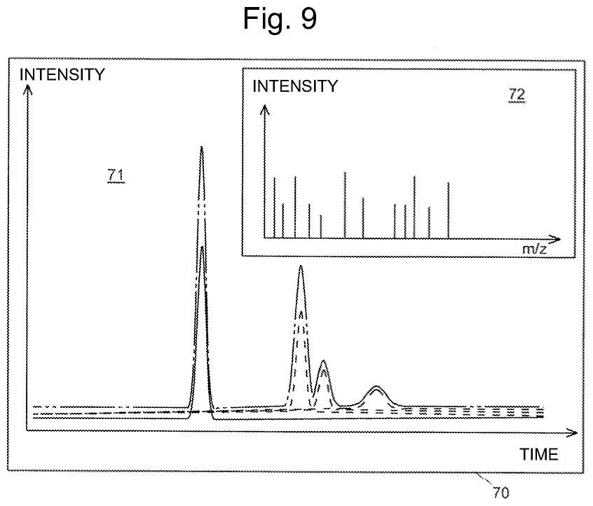

FIG. 7 shows one example of the merged MS/MS spectrum created by calculating an average of the intensities of mass peaks at each mass-to-charge ratio. FIG. 8 shows one example of the merged MS/MS spectrum created by extracting the highest value of the intensity of the mass peak at each mass-to-charge ratio. In FIG. 7, the two mass peaks indicated by the arrows correspond to the peaks which are present only in the MS/MS spectra of precursor ion 1. Therefore, calculating an average of the intensity values of those mass peaks results in their intensities being extremely low in the merged MS/MS spectrum. Although the mass spectrum schematically shown in FIG. 7 has a comparably small number of mass peaks, a merged MS/MS mass spectrum obtained through an actual measurement has a large number of mass peaks. Therefore, it is common practice to analyze only such mass peaks that exceed a threshold specified for distinguishing them from noise peaks. In that case, the mass peaks indicated by the arrows in FIG. 7 will be judged to be noise peaks and be excluded from the analysis, so that the fragment information which should actually be obtained from those mass peaks will not be obtained. On the other hand, in the merged MS/MS spectrum shown in FIG. 8, the two mass peaks indicated by the arrows, which are present only in the MS/MS spectra of precursor ion 1, also have sufficient intensities.

After the data of the merged MS/MS spectrum have been created for each compound, the compound determiner 46 extracts mass peaks which are present in the merged MS/MS spectrum of each compound (Step 7). The compound determiner 46 also theoretically calculates the mass-to-charge ratios of the fragment ions that can be generated from the compounds expected to be contained in the sample, and compares the calculated values with the mass-to-charge ratios of the extracted mass peaks to determine a fragment corresponding to each mass peak in the MS/MS spectrum (Step 8).

For example, in the case of a nucleic acid, which has a chain structure consisting of tens of nucleotides, such as adenine (A), guanine (G), cytosine (C), uracil (U) and thymine (T), it has been commonly known that the dissociation of a precursor ion occurs at a bonding site (“linker”) of those nucleotides. More specifically, it has been known that one of the following types of ions are generated as a product ion (fragment ion), depending on the dissociating location in the linker: a-ion, b-ion, c-ion, d-ion (on the 5′-terminal side), W-ion, X-ion, Y-ion and Z-ion (on the 3′-terminal side). Accordingly, it is possible to theoretically calculate the mass-to-charge ratios of the fragment ions that can be generated from a precursor ion in the MS/MS scan measurement. Similarly, the mass-to-charge ratios of the fragment ions resulting from foreign compounds can also be theoretically calculated from related information, such as the kind of reagent used for the chemical synthesis. By comparing those theoretically calculated mass-to-charge-ratio values with the mass-to-charge ratios of the mass peaks included in the data of the merged MS/MS spectrum, the fragment ion corresponding to each mass peak is determined, and the compound is identified (Step 9).

After the compounds contained in the sample have been identified, the analysis result displayer 47 shows an analysis result display screen on the display unit 6. FIG. 9 shows an example, in which the analysis result display screen 70 includes a chromatogram display section 71 and a mass spectrum display section 72.

The user issues a command to initiate a data analysis. Then, the analysis result displayer 47 shows the chromatogram display section 71 on the screen of the display unit 6. On the chromatogram display section 71, a total ion current chromatogram (TICC) for all compounds as well as a total ion current chromatogram (TICC) or extracted ion current chromatogram (XICC) for an individual compound can be displayed according to a user operation through the input unit 5.

On the chromatogram display section 71, the user indicates a specific point in time on one of the displayed chromatograms by using the input unit 5. Then, the mass spectrum display section 72 is additionally shown on the screen of the display unit 6. The mass spectrum display section 72 shows a mass spectrum acquired at the point in time indicated by the user on the chromatogram. For example, when a specific point in time is indicated on the TICC of all compounds, an MS spectrum acquired at that point in time (or within the cycle including that point in time) is displayed. When the TICC or XICC of an individual compound is selected, the merged MS/MS spectrum of that compound is displayed. FIG. 9 is an example in which the TICC of all compounds (long-dashed double short-dashed line) and the TICCs of individual compounds (solid or dashed line) are shown on the chromatogram display section 71, on which the user has selected the TICC of one compound (solid line), so that the merged MS/MS spectrum related to that compound is shown in the mass spectrum display section 72. The analysis result display screen 70 allows the user to check the temporal change in the amount of all compounds contained in the sample and the temporal change in the amount of each compound, as well as the information of the merged MS/MS spectrum of each compound. A section for showing information concerning the compound identified by the compound identifier 46 may also be displayed on the screen of the display unit 6 in addition to the chromatogram display section 71 and the mass spectrum display section 72 described in the present example.

When an analysis of product ions (fragment ions) generated from precursor ions having various number of charges is conducted for a sample which contains hundreds or even thousands of compounds, as with a chemically synthesized nucleic acid or peptide, if an attempt is made to analyze each individual set of data of an MS/MS spectrum obtained by an MS/MS scan measurement of each precursor ion, the number of sets of data will be enormous, and analyzing those data will require a considerable amount of time and labor.

By comparison, the method and device for processing mass spectrometry data according to the present embodiment only requires analyzing the data of one merged MS/MS spectrum for each compound. Therefore, the amount of time and labor required for the analysis can be dramatically reduced.

According to the present embodiment, the data of the merged MS/MS spectrum are created by extracting the highest value of the intensity of the mass peak at each mass-to-charge ratio from among the data of a plurality of MS/MS spectra acquired by MS/MS scan measurements. Therefore, the data of the merged MS/MS spectrum can be prepared without losing information concerning the mass peaks of product ions which are generated from only a specific precursor ion, or the mass peaks of product ions which appear with a low frequency.

The previous descriptions referring to FIGS. 5-8 were concerned with the case of acquiring data of MS/MS spectra by DDA. A similar problem can also occur in the case of acquiring data of MS/MS spectra by target MS/MS when a large number of compounds need to be subjected to the measurement within the same time period, since there may be a precursor ion for which an MS/MS scan measurement can be performed only within a time period where the amount of compound is small. Even in such a case, it is possible to create data of a merged MS/MS spectrum containing information on useful mass peaks, regardless of the timing of the execution of the MS/MS scan measurement, by extracting the highest value of the intensity of the mass peak in the data of the MS/MS spectra acquired within the time period concerned.

The previous embodiment is a mere example and can be appropriately changed or modified without departing from the spirit of the present invention.

Although a liquid chromatograph mass spectrometer was used in the previous embodiment, the previously described technique can also be similarly applied in the case of acquiring mass spectrometry data by a measurement using a gas chromatograph mass spectrometer or an independent mass spectrometer with no chromatograph. Although the ESI probe 211 was used for the ionization in the previous embodiment, any appropriate type of ion source can be used according to the characteristics of the sample. Furthermore, although a triple quadrupole mass spectrometer 20 was used in the previous embodiment, any type of mass spectrometer can be used which includes a mass separator having any configuration by which an MS/MS scan measurement can be performed.

In the previous embodiment, the data of the merged MS/MS spectrum were created by combining data of MS/MS spectra obtained from precursor ions which are identical in mass number and different in number of charges, on the assumption that the sample contains a nucleic acid or peptide. There are also other types of ions that can be included in the ions generated from the same compound (precursor ions), such as adduct ions ([M+X]⁺), isotopic ions, dehydrated ions ([M+H−H2O]⁺) and fragment ions (e.g., [Ma+H]⁺ and [Mb+H]⁺, where Ma+Mb=M). In the case of a sample from which any of these types of ions is expected to be generated, the ions to be classified as originating from the same compound can be determined from their respective mass-to-charge-ratio values, and data of the MS/MS spectra obtained by MS/MS scan measurements using those ions as precursor ions can be merged into one MS/MS spectrum.

### [Modes]

It is evident for a person skilled in the art that the previously described illustrative embodiment is a specific example of the following modes of the present invention.

### (Clause 1)

A method for processing mass spectrometry data according to one mode of the present invention includes the steps of:
preparing data of a plurality of MS/MS spectra for each of one or more compounds contained in a sample, each of the plurality of MS/MS being spectra acquired by an MS/MS scan measurement using each of a plurality of different precursor ions originating from the same compound; and
creating data of a merged MS/MS spectrum for each of the one or more compounds by merging the data of the plurality of MS/MS spectra acquired by the MS/MS scan measurements using the plurality of precursor ions originating from the compound concerned.

### (Clause 8)

A device for processing mass spectrometry data according to Clause 8 according to anther mode of the present invention includes:
a storage section holding data of a plurality of MS/MS spectra for each of one or more compounds contained in a sample, each of the plurality of MS/MS spectra being acquired by an MS/MS scan measurement using each of a plurality of different precursor ions originating from the same compound; and
a merged MS/MS spectrum data creator configured to create data of a merged MS/MS spectrum for each of the one or more compounds by merging the data of the plurality of MS/MS spectra acquired by the MS/MS scan measurements using the plurality of precursor ions originating from the compound concerned.

In the method and device for processing mass spectrometry data according to Clauses 1 and 8, data of a plurality of MS/MS spectra acquired by an MS/MS scan measurement using a plurality of different precursor ions originating from the same compound are prepared for each of the one or more compounds contained in a sample. This task may be carried out by acquiring the data by actual measurements, or by retrieving the data acquired beforehand. The plurality of different precursor ions include, for example, ions identical in mass number and different in number of charges, adduct ions, isotopic ions, dehydrated ions and fragment ions. By performing an MS/MS scan measurement using each ion as a precursor ion, data of a plurality of MS/MS spectra having different patterns can be obtained.

In the method and device for processing mass spectrometry data according to Clauses 1 and 8, data of a plurality of MS/MS spectra acquired in the previously described manner by an MS/MS scan measurement using precursor ions originating from the same compound are merged to obtain data of one merged MS/MS spectrum. The merging of the MS/MS spectra can be performed, for example, by calculating an average or total of the intensities of mass peaks having the same mass-to-charge ratios. The method and device for processing mass spectrometry data according to according to Clauses 1 and 8 only require checking the data of the merged MS/MS spectra whose number is equal to that of the compounds contained in the sample. Accordingly, the analyzing task can be more efficiently performed than by the conventional technique.

### (Clause 2)

The method for processing mass spectrometry data according to Clause 2, which is one mode of the method for processing mass spectrometry data according to Clause 1, further includes the step of identifying a compound by estimating a partial structure of the compound corresponding to a mass peak included in the data of the merged MS/MS spectrum, based on the mass-to-charge ratio of the mass peak.

By the method for processing mass spectrometry data according to Clause 2, a compound contained in the sample can be identified from the data of the merged MS/MS spectrum.

### (Clause 3)

In the method for processing mass spectrometry data according to Clause 3, which is one mode of the method for processing mass spectrometry data according to Clause 1 or 2, the data of the plurality of MS/MS spectra are data acquired by MS/MS scan measurements of a compound isolated by a column of a chromatograph.

In the method for processing mass spectrometry data according to Clause 3, different compounds are separated from each other by a column of a chromatograph. Therefore, even when ions whose mass-to-charge ratios are close to each other are generated from different compounds, it is possible to individually perform an MS/MS scan measurement of each of those ions and acquire data of an MS/MS spectrum.

### (Clause 4)

In the method for processing mass spectrometry data according to Clause 4, which is one mode of the method for processing mass spectrometry data according to Clause 3, the data of the merged MS/MS spectrum are created by collecting a mass peak having the highest intensity from among mass peaks having a common mass-to-charge ratio in the data of the plurality of MS/MS spectra.

In the method for processing mass spectrometry data according to Clause 3, since the amount of compound isolated by the column and subjected to the mass spectrometry changes with time, the measurement intensity of the ion varies depending on the timing at which the MS/MS scan is performed. Therefore, for example, if an average of the intensities of the mass peaks included in the data of a plurality of MS/MS mass spectra is calculated, the mass peaks included in the data of MS/MS spectra acquired at a timing where the amount of compound subjected to the mass spectrometry is small will have extremely low intensity values. In the method for processing mass spectrometry data according to Clause 4, the data of the merged MS/MS spectrum are created by collecting a mass peak having the highest intensity from among mass peaks having a common mass-to-charge ratio in the data of the plurality of MS/MS spectra. By this method, it is possible to create data of a merged MS/MS spectrum including mass peaks having sufficient intensities regardless of the timing at which the data of the MS/MS spectra were acquired.

### (Clause 5)

In the method for processing mass spectrometry data according to Clause 5, which is one mode of the method for processing mass spectrometry data according to Clause 3 or 4, the data of the plurality of MS/MS spectra are data acquired by DDA which includes repeating the process of performing an MS scan measurement of a compound isolated by the column to detect an ion having an intensity exceeding a previously determined threshold and perform an MS/MS scan measurement using the detected ion as a precursor ion.

In the method for processing mass spectrometry data according to Clause 5, all compounds contained in the sample are exhaustively subjected to the measurement by DDA. Therefore, even when a large number of compounds are expected to be contained in the sample, data of the MS/MS spectra of the large number of compounds can be acquired without requiring the MS/MS scan measurement condition to be set for each of the large number of compounds. The method for processing mass spectrometry data according to Clause 5 can be suitably used for the measurement of a sample containing a large number of foreign compounds other than the target compounds, as with a sample of a chemically synthesized nucleic acid or peptide.

### (Clause 6)

In the method for processing mass spectrometry data according to Clause 6, which is one mode of the method for processing mass spectrometry data according to Clause 5, the data of the merged MS/MS spectrum are created by merging data of a plurality of MS/MS spectra acquired by an MS/MS scan measurement using precursor ions which are identical in mass number and different in number of charges.

Ionizing a nucleic acid or peptide produces ions having different numbers of charges. When ions (precursor ions) which are identical in molecular structure yet different in number of charges are dissociated, each precursor ion may be dissociated at a different position and produce different fragments, yielding data of an MS/MS spectrum with a different pattern. In the method for processing mass spectrometry data according to Clause 6, data of MS/MS spectra acquired for precursor ions having different numbers of charges are merged to create data of a merged MS/MS spectrum which includes mass peaks corresponding to various fragments, whereby compounds can be identified with a high level of accuracy.

### (Clause 7)

The method for processing mass spectrometry data according to Clause 7, which is one mode of the method for processing mass spectrometry data according to one of Clauses 3-6, further includes the step of displaying, on a screen, a chromatogram and the merged MS/MS spectrum for each compound contained in the sample.

The method for processing mass spectrometry data according to Clause 7 allows the user to visually check a chromatogram and MS/MS spectrum of a compound contained in a sample on the screen. When this mode is combined with the method for processing mass spectrometry data according to Clause 2, an identification result of the compound can also be displayed on the screen, which allows the user to additionally check the identification result on the screen.

### REFERENCE SIGNS LIST

1 . . . Liquid Chromatograph Mass Spectrometer
10 . . . Liquid Chromatograph
11 . . . Mobile Phase Container
12 . . . Pump
13 . . . Injector
14 . . . Column
15 . . . Mass Spectrometer
21 . . . Ionization Chamber
211 . . . ESI Probe
22 . . . First Intermediate Vacuum Chamber
221 . . . Ion Guide
23 . . . Second Intermediate Vacuum Chamber
231 . . . Ion Guide
24 . . . Third Intermediate Vacuum Chamber
241 . . . Quadrupole Mass Filter
243 . . . Collision Cell
244 . . . Multipole Ion Guide
245 . . . Ion Guide
25 . . . Analysis Chamber
251 . . . Ion Guide
252 . . . Orthogonal Acceleration Electrode
2521 . . . Push-Out Electrode
2522 . . . Pulling Electrode
253 . . . Second Acceleration Electrode
254 . . . Reflectron
2541 . . . First Reflectron
2542 . . . Second Reflectron
255 . . . Ion Detector
256 . . . Flight Tube
257 . . . Back Plate
40 . . . Control-and-Processing Unit
41 . . . Storage Section
42 . . . Measurement Condition Setter
43 . . . Measurement Executer
44 . . . Precursor Ion Classifier
45 . . . Merged MS/MS Spectrum Data Creator
46 . . . Compound Identifier
47 . . . Analysis Result Displayer
5 . . . Input Unit
6 . . . Display Unit
70 . . . Analysis Result Display Screen
71 . . . Chromatogram Display Section
72 . . . Mass Spectrum Display Section
C . . . Ion Beam Axis

1. A method for processing mass spectrometry data, comprising steps of:
preparing data of a plurality of MS/MS spectra for each of one or more compounds contained in a sample, each of the plurality of MS/MS spectra being acquired by an MS/MS scan measurement using each of a plurality of different precursor ions originating from the same compound; and
creating data of a merged MS/MS spectrum for each of the one or more compounds by merging the data of the plurality of MS/MS spectra acquired by the MS/MS scan measurements using the plurality of precursor ions originating from the compound concerned.

2. The method for processing mass spectrometry data according to claim 1, further comprising a step of identifying a compound by estimating a partial structure of the compound corresponding to a mass peak included in the data of the merged MS/MS spectrum, based on a mass-to-charge ratio of the mass peak.

3. The method for processing mass spectrometry data according to claim 1, wherein the data of the plurality of MS/MS spectra are data acquired by MS/MS scan measurements of a compound isolated by a column of a chromatograph.

4. The method for processing mass spectrometry data according to claim 3, wherein the data of the merged MS/MS spectrum are created by collecting a mass peak having a highest intensity from among mass peaks having a common mass-to-charge ratio in the data of the plurality of MS/MS spectra.

5. The method for processing mass spectrometry data according to claim 3, wherein the data of the plurality of MS/MS spectra are data acquired by DDA which includes repeating a process of performing an MS scan measurement of a compound isolated by the column to detect an ion having an intensity exceeding a previously determined threshold and perform an MS/MS scan measurement using the detected ion as a precursor ion.

6. The method for processing mass spectrometry data according to claim 5, wherein the data of the merged MS/MS spectrum are created by merging data of a plurality of MS/MS spectra acquired by MS/MS scan measurements using precursor ions which are identical in mass number and different in number of charges.

7. The method for processing mass spectrometry data according to claim 3, further comprising a step of displaying, on a screen, a chromatogram and the merged MS/MS spectrum for each compound contained in the sample.

8. A device for processing mass spectrometry data, comprising:
a storage section holding data of a plurality of MS/MS spectra for each of one or more compounds contained in a sample, each of the plurality of MS/MS spectra being acquired by an MS/MS scan measurement using each of a plurality of different precursor ions originating from the same compound; and
a merged MS/MS spectrum data creator configured to create data of a merged MS/MS spectrum for each of the one or more compounds by merging the data of the plurality of MS/MS spectra acquired by the MS/MS scan measurements using the plurality of precursor ions originating from the compound concerned.
